(19) United States
(12) Patent Application Publication
(10) Pub. No.: US 2008/0241265 A1
(43) Pub. Date: Oct. 2, 2008

(54) Pharmaceutical Combinations Containing Lamivudine, Stavudine and Nevirapine

(76) Inventors: Amar Lulla, Maharashtra (IN); Geena Malhotra, Maharashtra (IN)

Correspondence Address: CONLEY ROSE, P.C., 5601 GRANITE PARKWAY, SUITE 750, PLANO, TX, 75024, US

(21) Appl. No.: 12/065,367

(22) PCT Filed: Aug. 31, 2006
(86) PCT No.: PCT/GB06/03229

§ 371 Date: May 9, 2008

(30) Foreign Application Priority Data
Aug. 31, 2005 (IN) 1057/MUM/2005

(51) Int. Cl. A61K 9/14; A61K 31/7072; A61P 31/18

(52) U.S. Cl. 424/490; 514/50

(57) ABSTRACT

A pharmaceutical composition comprising 10-120 mg lamivudine, 1-30 mg stavudine and 50-170 mg nevirapine for pediatric treatment of viral infections. One particularly preferred composition comprises a tablet containing 12 mg stavudine, 60 mg lamivudine and 100 mg nevirapine. Another particularly preferred composition comprises a second tablet containing 6 mg stavudine, 30 mg lamivudine and 500 mg nevirapine. These compositions are suitable for treating children having a body weight from 5 to 30 kg.

### CROSS-REFERENCE TO RELATED APPLICATIONS

This application is a filing under 35 U.S.C. 371 of International Application No. PCT/GB2006/003229 filed Aug. 31, 2006, entitled “Pharmaceutical Combinations Containing Lamivudine, Stavudine and Nevirapine,” claiming priority of Indian Patent Application No. 1057/MUM/2005 filed Aug. 31, 2005, which applications are incorporated by reference herein in their entirety.

### FIELD OF THE INVENTION

The present invention relates to a pharmaceutical composition and a method of inhibiting human immunodeficiency virus (HIV) comprising the preparation and administration of a homogenous combination of lamivudine, stavudine and nevirapine to an HIV infected patient in an amount which achieves antiviral efficacy.

### BACKGROUND OF THE INVENTION

A retrovirus designed human immunodeficiency virus (HIV) is the etiological agent of the complex disease that includes progressive destruction of the immune system (acquired immune deficiency syndrome or AIDS) and degeneration of the central and peripheral nervous system.

A common feature of retrovirus replication is the extensive post-translation processing of precursor polyproteins by a virally encoded protease to generate mature viral proteins required for virus assembly and function. Inhibition of this processing prevents the production of normally infectious virus. Literature reports that genetic inactivation of the HIV encoded protease resulted in the production of immature, non-infectious virus particles. These results indicate that inhibition of the HIV protease represents a viable method for the treatment of AIDS and the prevention or treatment of infection by HIV.

Nucleotide sequencing of HIV shows the presence of a Dol gene in one open reading frame [as reported in ‘Nature’, 313, 277 (1985) by Ratner, L. et al]. Amino acid sequence homology provides evidence that the Dol sequence encodes reverse transcriptase, an endonuclease and an HIV protease [Toh, H. et al., EMBO J., 4, 1267 (1985); Power, M. D. et al., Science, 231, 1567 (1986); Pearl, L. H. et al., Nature, 329, 351 (1987)].

U.S. Pat. No. 6,486,183 relates to the field of antivirals and in particular to HIV reverse transcriptase inhibitors and provides novel compounds, pharmaceutical compositions comprising these compounds and methods for the inhibition of HIV employing them.

WO2004087169 relates to an invention which provides for a pharmaceutical composition useful for the treatment or prophylaxis of viral infections comprising nevirapine and at least one antiviral active compound, wherein Base is selected from the group consisting of thymine, cytosine, adenine, guanine, inosine, uracil, 5-ethyluracil and 2,6-diaminopurine, or a pharmaceutically acceptable salt or prodrug thereof, an example of such antiviral active compound being alovudine.

Lamivudine has proven antiviral activity against human immunodeficiency virus (HIV) and other viruses such as hepatitis B. Lamivudine is commercially available from Glaxo Wellcome Inc under trade name EPIVIR. Lamivudine and its use against HIV are described in WO 91/17159 and EP 0382526. Crystalline forms of lamivudine are described in WO 92/21676. Combinations of lamivudine with other reverse transcriptase inhibitors, in particular zidovudine, are described in, for example, WO 92/20344, WO 98/18477, and WO99/55372.

U.S. Pat. No. 5,047,407 discloses (2R, cis)-4-ammino-1-(2-hydroxymethyl-1,3-oxathiolan-5-yl)-(1H)-pyrimidin-2-one (Epivir—RTM., Lamivudine) and its use in the treatment and prophylaxis of viral infections. Lamivudine has proven antiviral activity against HIV and other viruses such as HBV.

Stavudine, a nucleoside reverse transcriptase inhibitor, and its preparation are disclosed, for example, in U.S. Pat. No. 4,978,655. It is known that stavudine is effective in the treatment of infections caused by retroviruses such as murine leukemia virus and human immunodeficiency virus, i.e. HIV; HTLV III/LAV virus (the AIDS virus).

Stavudine is commercially available from Bristol Myers Squibb Co. under the trademark Zerit™ for treatment of HIV as described in U.S. Pat. No. 4,978,655. Methods of preparation of Stavudine are also described in, for example, AU8519701, WO02/20538, US2001039342 and WO01/77103.

The formulations containing stavudine are further described in, for example, US2002002147, WO017/4329, FR2794752 and AU4959101.

Nevirapine is commercially available from Boehringer Ingelheim under the trademark Viramune for treatment of HIV as described in U.S. Pat. No. 6,172,059 and U.S. Pat. No. 6,255,481 and in WO02/092095. The earliest known synthesis of nevirapine, by Hargrave et al, is described in U.S. Pat. No. 5,366,972.

Another patent EA4767 relates to a combination useful for the treatment of viral infections comprising at least one compound wherein the nucleoside analogue is chosen from zidovudine, didanosine, zalcitabine, stavudine or lamivudine and the non-nucleoside reverse transcriptase inhibitor is chosen from nevirapine, delavirdine or efavirenz and wherein the protease inhibitor is chosen from indinavir, nelfinavir, saquinavir or ritonavir. It also deals with a method for the treatment of viral infections comprising administering a therapeutically effective amount of a compound to a subject suffering from an HIV infection.

One substantial and persistent problem in the treatment of AIDS has been the ability of the HIV virus to develop resistance to the individual therapeutic agents employed to treat the disease. Thus, a need remains for an efficacious and long lasting therapy for AIDS which lowers HIV viral levels of patients to undetectable levels and raises CD4 cell counts for prolonged periods of time without the development of resistance.

### SUMMARY OF THE INVENTION

It is an object of the present invention to provide a pharmaceutical composition, which, inter alia, will assist in inhibiting the human immunodeficiency virus (HIV).

The present invention relates to pharmaceutical compositions for treating human immunodeficiency virus (HIV) infections.

An object of the present invention is to provide a pharmaceutical composition comprising at least two nucleoside reverse transcriptase inhibitors or pharmaceutically acceptable salts and esters thereof, combined together for co-administration with at least one non-nucleoside reverse transcriptase inhibitor or pharmaceutically acceptable salts and esters thereof in a pharmaceutical acceptable carrier or excipient.

A further object of the present invention is to provide a method for the manufacture of the pharmaceutical composition according to the present invention.

A further object of the present invention is to provide a combination therapy as a method to enhance the effectiveness in treating AIDS and to preclude the development of resistance to individual therapeutic agents.

Yet another object of the present invention is to provide a method for treating, reversing, reducing or inhibiting retroviral infections, in particular HIV infections in a human, which includes administering to a mammal a safe and effective amount of the pharmaceutical composition as set out herein.

Yet another object of the present invention, use of a composition for the treatment of viral infections, particularly retroviral infections, which may include human immunodeficiency virus (HIV) infections.

The present invention provides a pharmaceutical composition comprising at least two nucleoside reverse transcriptase inhibitors (“NRTI”) or pharmaceutically acceptable salts and esters thereof combined together for co-administration with at least one maturation inhibitor, antisense compound and/or non-nucleoside reverse transcriptase inhibitor (nNRTI) or pharmaceutically acceptable salts and esters thereof in a pharmaceutical acceptable carrier or excipient.

According to the present invention there is also provided a method of manufacturing a pharmaceutical composition comprising combining at least two NRTIs or pharmaceutically acceptable salts and esters thereof together with at least one maturation inhibitor, antisense compound and/or nNRTI or pharmaceutically acceptable salts and esters thereof, and a pharmaceutically acceptable carrier.

According to the present invention there is also provided combination therapy as a method to enhance the effectiveness in treating AIDS and to preclude the development of resistance to individual therapeutic agents.

### DETAILED DESCRIPTION OF THE INVENTION

In the present invention, two or more NRTIs or pharmaceutically acceptable salts and esters thereof are combined together for co-administration with at least one maturation inhibitor, antisense compound and/or nNRTI or pharmaceutically acceptable salts and esters thereof. This combination therapy is a method to enhance the effectiveness in treating AIDS and to preclude the development of resistance to individual therapeutic agents.

The NRTIs are preferably selected from at least two of Abacavir Sulfate, Didanosine, Emtricitabine, Lamivudine, Stavudine, Tenofovir disoproxil fumarate, Zalcitabine, Amdoxovir, Elvucitabine, GS-7340, INK-20 (thioether phospholipid formulation of AZT), MIV-310, MIV-210, Racivir, Reverset, Zidovudine, SPD-754, BCH-13520, BCH-10618 or pharmaceutically acceptable salts, esters or prodrugs thereof. Most preferably the NRTIs include Lamivudine and stavudine, or pharmaceutically acceptable salts, esters or prodrugs thereof.

The further antiviral agent may be selected from the group of the maturation inhibitors, antisense compounds or nNRTIs. Examples of further antivirals include PA-457, KPC-2, HGTV-43, Delavirdine, Efavirenz, (+)-Calanolide A and B, Capravirine, nevirapine, GW-695634, MIV-150, MV026048, NV-05, R-278474, RS-1588, TMC-120/125, TMC-125, UC-781, YM-215389 or pharmaceutically acceptable salts, esters or prodrugs thereof. Most preferably the further antiviral agent is nevirapine or pharmaceutically acceptable salts, esters or prodrugs thereof.

It will be appreciated that the pharmaceutical combinations according to the invention may be combined with further active ingredients.

Examples of such further active ingredients are acyclic nucleosides such as acyclovir, ganciclovir; interferons such as alpha-, beta- and gamma-interferon; glucuronation inhibitors such as probenecid; nucleoside transport inhibitors such as dipyridamole; immunomodulators such as interleukin II (IL2) and granulocyte macrophage colony stimulating factor (GM-CSF), erythropoietin, ampligen, thymomodulin, thymopentin, foscamet, glycosylation inhibitors such as 2-deoxy-D-glucose, castanospermine, 1-deoxynojirimycin; and inhibitors of HIV binding to CD4 receptors such as soluble CD4, CD4 fragments, CD4-hybrid molecules and inhibitors of the HIV aspartyl protease such as L-735, 524.

HIV causes a variety of clinical conditions including acquired immunodeficiency syndrome (AIDS) and chronic neurological disorders. Single drug treatment regimens typically require long term treatment increasing the evidence of unwanted side effects. Moreover, single drug therapies are particularly vulnerable to mutation in the HIV runs, leading to drug resistant variants of HIV.

Multiple drug regimes dramatically improve the treatment of HIV infected patients. This is because one drug will usually cancel out mutations against other drugs. Multiple drug therapies even inhibit replication of HIV viruses for a period of time sufficient to eliminate HIV from the body.

The success of modern multiple drug treatments for HIV often requires strict compliance with a complex treatment regimen that can require the administration of many different drugs per day, administered at precisely times intervals with careful attention to diet. Patient non-compliance is a well-known problem accompanying such complex treatment regimens. Patient non-compliance is an important problem in the treatment of HIV because such non-compliance may lead to the emergence of multiple drug resistant strains of HIV.

This combination therapy in accordance with the invention provides a method to enhance the effectiveness in treating AIDS and to preclude the development of resistance to the individual therapeutic agents.

In general, during alternation therapy, an effective dosage of each agent is administered serially, whereas in combination therapy, an effective dosage of two or more agents are administered together. The dosages will depend on such factors as absorption, biodistribution, metabolism and excretion rates for each drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. Examples of suitable dosage ranges for nevirapine, preferably 3′-deoxy-3′-fluorothymidine, further NRTIs and other antivirals can be found in the scientific literature. Many examples of suitable dosage ranges for other compounds described herein are also found in the public literature or can be identified using known procedures. These dosage ranges can be modified as desired to achieve a desired result.

The compositions according to the invention may be administered as often as necessary to achieve the desired therapeutic effect. In practice, the compositions may be administered, for example, once, twice, three times or four times per day; other they may be administered less than once per day, for example once every two days or once per week.

The active ingredients can be administered orally in solid dosage forms, such as capsules, tablets and powders, or in liquid dosage forms, such as elixirs, syrups and suspensions. According to the invention, a pharmaceutical composition may include in combination Lamivudine, Stavudine and nevirapine, or pharmaceutically acceptable derivatives thereof as a solid oral dosage form, preferably as a tablet.

The dosage form according to the invention is preferably a dispersible dosage form. Dispersable tablets rapidly disintegrate in cold water (i.e. water of a temperature from about 5° C. to about 30° C.) to produce a suspension suitable for ingestion. Dispersable tablets have a number of advantages, in particular, they are easy to administer in pediatric applications, and they are easy to manufacture and store. Therefore, the dosage form preferably includes a disintegrant.

The formulations may be prepared by any of the methods well known in the art of pharmacy. Pharmaceutical formulation suitable for oral administration may conveniently be presented as discrete units such as capsules, including soft gelatin capsules, cachets or tablets each containing a predetermined amount of the active ingredient (s); as a powder or granules. Tablets and capsules for oral administration may contain conventional excipients such as binding agents, fillers, lubricants, disintegrants, or wetting agents. The tablets may be coated according to methods well known in the art.

The combination therapy may include a weight ratio of nevirapine to Lamivudine ranging from about 6:3 to 4:3, most preferably about 5:3; and the weight ratio of Lamivudine to Stavudine is preferably about 6:1 to 4:1, most preferably about 5:1.

In these embodiments, a pharmaceutically acceptable salt or ester can be substituted for any one or more of the compounds per se. In yet another aspect of these methods, the weight ratio of Nevirapine to Lamivudine and Stavudine can be any one of the weight ratios set forth above.

Exemplifying the invention is a method of preventing infection by HIV, or of treating infection by HIV, or of preventing or treating AIDS, comprising administering to a subject in need thereof a therapeutically effective amount of the compositions described above.

Further exemplifying this invention is the use of Nevirapine and two or more antiretroviral agents selected from nucleoside reverse transcriptase inhibitors such as Stavudine, Lamivudine, Zidovudine or pharmaceutically acceptable derivatives thereof, in the preparation of a medicament for the treatment of infection by HIV and/or for the treatment of AIDS which comprises an effective amount of above mentioned antiretroviral agents or pharmaceutically acceptable derivatives thereof, together or separately. Additionally, the present invention includes a process for making a pharmaceutical composition comprising combination of nucleoside reverse transcriptase inhibitors and nonnucleoside reverse transcriptase inhibitors as mentioned above and a pharmaceutically acceptable carrier.

More specifically exemplifying the invention is a pharmaceutical composition comprising nevirapine, Stavudine and Lamivudine, and pharmaceutically acceptable derivatives thereof, in a pharmaceutically acceptable carrier.

Lamivudine (also known as 3TC) is a synthetic analogue, chemically known as (2R-cis)-4-Amino-1-[2-(hydroxymethyl) 1,3-oxathiolan-5-yl]-2(1H)-pyrimidinone. Lamivudine has also been referred to as (−)-1-[(2R,5S) 2-(Hydroxymethyl)-1,3-oxathiolan-5-yl]cystosine, (Hydroxymethyl)-1,3-oxathiolan-5-yl]cystosine.

It has been found that Lamivudine exhibits unexpected advantages when used in combination with known inhibitors of HIV replication. In particular, lamivudine shows a better antiviral effect when used in combination with Stavudine.

Stavudine chemically known as (3′-deoxythymidin-2′-ene(3′-deoxy-2′,3′-didehydrothymidine), is the synthetic thymidine nucleoside, now well established as an important and useful chemotherapeutic agent for the therapeutic treatment of patients infected with retroviruses.

The chemical name for nevirapine is 11-cyclopropyl-5,11-dihydro-4-methyl-6H-dipyrido[3,2-b: 2′,3′-e][1,4]diazepin-6-one. Nevirapine is an HIV-1 specific, non-nucleoside, reverse transcriptase inhibitor (NNRTI). Nevirapine is used for treatment of HIV. It is reported to inhibit reproduction of HIV in the body. Nevirapine is used in conjunction with other retroviral agents.

Interestingly, it has been found that nevirapine when used in antiretroviral regimens that include nucleoside reverse transcriptase inhibitors like lamivudine and stavudine has been found to be very effective.

By means of the pharmaceutical composition in accordance with the invention treatment regimens for HIV and other viruses can be simplified with the goal of enhancing patient compliance by providing a simplified dosage therapy containing a combination of pharmaceutically acceptable amounts of Lamivudine, Stavudine and Nevirapine or pharmaceutically acceptable derivatives thereof.

The phrase ‘pharmaceutically acceptable derivative’ as used herein is intended to any pharmaceutically acceptable salt, enantiomer, solvent, ester or salt of such ester, or any other compound or mixture which, upon administration to the recipient, is capable of providing (directly or indirectly) the intended active ingredient or any active metabolite or residue thereof.

In the most preferred embodiment, the pharmaceutical composition of the invention employs a combination safe and therapeutically effective amount of two or more therapeutically active agents viz. safe and therapeutically effective amounts of Nevirapine, or 11-cyclopropyl-5,11-dihydro-4-methyl-6H-dipyrido[3,2-b:2′,3′-e][1,4]diazepin-6-one and its pharmaceutically acceptable salts, solvents and derivatives thereof, a safe and therapeutically effective amounts (−) 2′,3′-dioxy, 3′-thyacytidine (Lamivudine) or its pharmaceutically acceptable salts, solvents and derivatives thereof, a safe and therapeutically effective amounts of Stavudine, (3′-deoxythymidin-2′-ene(3′-deoxy-2′,3′-didehydrothymidine), or its pharmaceutically acceptable salts, solvents and derivatives thereof along with a safe and effective amount of pharmaceutically acceptable excipients to maintain the composition's homogeneity prior to tablet compression.

The pharmaceutical composition of the present invention conveniently allows administration of a pharmaceutical kit containing three active compounds in tablet dosage forms containing specific dosage ranges for each compound.
Lamivudine of about 100-300 mg per unit dosage form
Stavudine of about 30-100 mg per unit dosage form
Nevirapine of about 150-400 mg per unit dosage form.

According to the present invention, the pharmaceutical composition is for pediatric use and dosage regimen is accordingly adjusted. Dosage ranges for each compound for pediatric use is advantageously in the following range:
Lamivudine of about 10-120 mg more preferably about 60 mg per unit dosage form
Stavudine of about 1-30 mg more preferably about 12 mg per unit dosage form
Nevirapine of about 25-170 mg, more preferably 50-170 mg, more preferably about 100 mg per unit dosage form.

Therefore, according to another aspect of the invention there is provided a pharmaceutical composition comprising lamivudine, stavudine, and nevirapine, or pharmaceutically acceptable salts or esters thereof, for separate, simultaneous or sequential administration, comprising 10-120 mg lamivudine, 1-30 mg stavudine and 25-170 mg nevirapine.

Preferably, the pharmaceutical composition comprises 10-100 mg lamivudine, 1-25 mg stavudine and 25-170 mg nevirapine.

Preferably, the wt % of nevirapine in the composition is from 0.75 to 2.0 times, more preferably 0.75 to 1.5 times, the wt % of lamivudine in the composition. Preferably, the wt % of lamivudine in the composition is from 2 to 6 times, the wt % of stavudine in the composition.

The pharmaceutical composition is most preferably a single unit dosage form containing said lamivudine, stavudine and nevirapine. The dosage form is desirably a solid oral dosage form.

One particularly preferred composition comprises a tablet containing 12 mg stavudine, 60 mg lamivudine and 100 mg nevirapine. This is referred to as tablet 1 in the table below.

Another particularly preferred composition comprises a second tablet containing 6 mg stavudine, 30 mg lamivudine and 50 mg nevirapine. This is referred to as tablet 2 in the table below.

This composition is particularly useful for pediatric use i.e for treating human under the age of 16 years. The preferred dosage depends on the body weight of the child to be treated. The following dosages are most suitable:

| | | | | | Number |
|---|---|---|---|---|---|
| Body | Stavudine/ | lamivudine/ | Nevirapine/ | Number of | of |
| Weight/kg | mg | mg | mg | Tablet 1 | Tablet 2 |
| 3-5 | 3 | 15 | 25 | | 0.5 |
| 6-7 | 6 | 30 | 50 | | 1 |
| 8-9 | 9 | 45 | 75 | | 1.5 |
| 10-14 | 12 | 60 | 100 | 1 | |
| 15-19 | 15 | 75 | 125 | | 2.5 |
| 20-24 | 18 | 90 | 150 | 1.5 | 3 |
| 25-29 | 24 | 120 | 200 | 2 | |

Compatibility was another aspect which had to be sorted for these 3 drugs. Study was designed so as to evaluate the physico-chemical parameters of these three drugs. Each active ingredients was homogeneously mixed with the other and after a period of four weeks exposure to 25° C./60% RH & 40° C./75% RH, this mixture was analysed.

The details are enclosed below:

| | Appearance | | Impurities | |
|---|---|---|---|---|
| | | End of | | End of |
| Ingredient | Initial | study | Initial | study |
| Lamivudine + Nevirapine | White | White | 0.54% | 0.58% |
| Lamivudine + Stavudine | White | Brown | 0.87% | 1.25% |
| Stavudine + Nevirapine | White | Brown | 0.95% | 1.97% |
| Lamivudine + Nevirapine + | White | Brown | 1.28% | 12.35% |
| stavudine | | | | |

Lamivudine was compatible with Nevirapine but not with Stavudine. Similarly, stavudine was incompatible with nevirapine.

Incompatibility with Lamivudine & Nevirapine can be avoided by separating stavudine from the other two drugs by one of the following ways:
i. Coating of Stavudine or Stavudine granules by suitable film forming polymers & adding to granules at lubrication stage;
ii. By employing bilayer/trilayer design for the tablets.

The tablet according to the present invention may be prepared by using standard methods of tablet manufacture i.e. wet granulation or direct compression. Coating techniques for the stavudine granules employs methods and equipments which are extensively documented in literature.

The formulation may further comprise binders, diluents, disintegrants, glidants, lubricants and artificial colours. The binders are usually used the ranges of 0.5 to 25%, disintegrants in the range of 0.5-25%, lubricants in the range of 0.25%-10%.

A tablet may also contain some pharmaceutically acceptable fillers as excipients. Examples of suitable fillers/diluents are starch and derivatives, lactose, mannitol, sucrose, glucose, Sorbitol, calcium phosphates, maltodextrins, polyvinylpyrrolidone, polyethylene glycols, microcrystalline cellulose, etc.

The tablets according to the present invention may also contain other excipients like binders (microcrystalline cellulose, starches, polyvinylpyrrolidone and the like), disintegrants (microcrystalline cellulose, sodium starch glycollate, starch, croscarmellose sodium, hydroxypropyl cellulose, etc.), lubricants (talc, Magnesium stearate, colloidal silica and the like), flavouring or colouring agents.

i. Option I
Stavudine API as a powder or in form of granules may be coated using suitable film forming polymers such as HPMC, HPMCP, Na-CMC, HPC, PVA, PVP, acrylates such as Eugragit to E-100, L-100, L10055, HPMC acetyl succinate, Xanthan gum etc. Coated Stavudine may be blended with granules of Lamivudine & Nevirapine along with lubricants & compressed using suitable tooling.
   ii. Option II
a. To manufacture bilayered tablet or trilayeral where in stavudine is incorporated as one of the layer & lamivudine & Nevirapine in the 2^{nd} layer or as a trilayered tablet where in Stavudine layer & lamivudine & Nevirapine layer separated totally by an inert layer.
b. Tablet in a tablet is another option available where in Stavudine layer constitutes inner tablet which may be film coated or uncoated.

Therefore, in a preferred embodiment, a barrier is provided between said stavudine and said nevirapine and lamivudine.

In one preferred embodiment, said stavudine is provided in the form of particles coated with a material to prevent contact between said stavudine and said nevirapine and lamivudine.

In another preferred embodiment the pharmaceutical composition comprises two layers, wherein the first layer contains said nevirapine and lamivudine in combination with a pharmaceutically acceptable carrier, and the second layer contains said stavudine in combination with a pharmaceutically acceptable carrier.

In another preferred embodiment the pharmaceutical composition comprises three layers, wherein the first layer contains said nevirapine and lamivudine in combination with a pharmaceutically acceptable carrier, the second layer contains said stavudine in combination with a pharmaceutically acceptable carrier, and the third layer is an inert layer containing at least one pharmaceutically acceptable excipient, wherein the third layer is disposed between the first and second layers.

In another preferred embodiment the pharmaceutical composition comprises a core and an outer layer surrounding the core, wherein the core contains said nevirapine and lamivudine in combination with a pharmaceutically acceptable carrier, and outer layer contains said stavudine in combination with a pharmaceutically acceptable carrier.

In another preferred embodiment the pharmaceutical composition comprises a core and an outer layer surrounding the core, wherein the core contains said stavudine in combination with a pharmaceutically acceptable carrier, and outer layer contains said nevirapine and lamivudine in combination with a pharmaceutically acceptable carrier.

According to another aspect of the invention there is provided the use of a lamivudine, nevirapine and stavudine in the manufacture of a medicament for treatment of a viral infection in a human, wherein said medicament contains 10-120 mg lamivudine, 1-30 mg stavudine and 25-170 mg nevirapine.

The viral infection may be a retroviral infection, such as HIV infection.

According to another aspect of the invention, there is provided a method of treating a viral infection in a human, comprising administering to a human in need thereof an amount of a medicament containing 10-120 mg lamivudine, 1-30 mg stavudine and 25-170 mg nevirapine at therapeutically acceptable intervals.

According to another aspect of the invention, there is provided a method of making a pharmaceutical composition comprising combining 10-120 mg lamivudine, 1-30 mg stavudine and 25-170 mg nevirapine with a pharmaceutically acceptable carrier.

In one aspect, the invention provides a way of providing a single, stable, oral dosage form comprising stavudine, lamivudine and nevirapine.

According to another aspect of the invention there is provide a pharmaceutical composition in the form of an oral dosage form comprising stavudine, lamivudine and nevirapine, wherein the lamivudine and nevirapine are provided in one layer of the oral dosage form, and the stavudine is provided in a separate layer. The dosage form can be provided in the bilayer or trilayer form described above, and may be made by the methods described above.

According to another aspect of the invention there is provided a pharmaceutical composition in the form of an oral dosage form comprising stavudine, lamivudine and nevirapine, wherein said stavudine is provided in the form of particles coated with a material to prevent contact between said stavudine and said nevirapine and lamivudine.

The following examples are for the purpose of illustration of the invention only and are not intended in any way to limit the scope of the present invention.

### EXAMPLE 1

| Ingredients | Qty (mg/tablet) |
|---|---|
| Stavudine | 12.00 |
| HPMC | 3.00 |
| Talc | 1.00 |
| Purified water | q.s. |
| Nevirapine | 100.00 |
| Lamivudine | 60 |
| Microcrystalline cellulose (Avicel PH 101) | 237.50 |
| Sodium starch glycollate | 25.00 |
| Starch | 10.00 |
| Sodium starch glycollate | 10.00 |
| Magnesium stearate | 7.50 |

### EXAMPLE 2

| Ingredients | Qty (mg/tablet) |
|---|---|
| Stavudine | 12.00 |
| HPC-L | 2.00 |
| Propylene glycol | 0.2 |
| Purified water | q.s. |
| Nevirapine | 100.00 |
| Lamivudine | 60 |
| Microcrystalline cellulose (Avicel PH 101) | 237.50 |
| Sodium starch glycollate | 25.00 |
| Starch | 10.00 |
| Sodium starch glycollate | 10.00 |
| Magnesium stearate | 7.50 |

### EXAMPLE 3

| Ingredients | Qty (mg/tablet) |
|---|---|
| Stavudine | 12.0 |
| Eudragit L100 | 7.0 |
| Dibutyl phthalate | 0.7 |
| Isopropyl alcohol | q.s. |
| Purified water | q.s. |
| Nevirapine | 100.00 |
| Lamivudine | 60 |
| Microcrystalline cellulose (Avicel PH 101) | 237.50 |
| Sodium starch glycollate | 25.00 |
| Starch | 10.00 |
| Sodium starch glycollate | 10.00 |
| Magnesium stearate | 7.50 |

### EXAMPLES

### 1. Trilayered:

| | Qty | |
|---|---|---|
| Ingredients | (mg/tablet) | |
| Part I | | |
| Lamivudine | 60.00 | |
| Nevirapine | 100.00 | |
| Microcrystalline cellulose | 73.00 | |
| Starch | 6.00 | |
| Sodium Starch glycollate | 8.00 | |
| Talc | 1.00 | |
| Magnesium Stearate | 2.00 | |
| Purified water | q.s. | |
| | 250.00 | |
| Part II | | |
| Microcrystalline cellulose | 69.95 | |
| Starch | 4.00 | |
| Sodium Starch glycollate | 4.00 | |
| Colourant | 0.05 | |
| Purified water | q.s. | |
| Talc | 1.00 | |
| Magnesium stearate | 1.00 | |
| | 80.00 | |
| Part III | | |
| Stavudine | 12.00 | |
| Mannitol | 81.0 | |
| Hydroxypropyl cellulose | 4.00 | |
| Aerosil | 0.6 | |
| Talc | 1.2 | |
| Magnesium Stearate | 1.2 | |
| | 100.00 | |
| Total | 430.0 | mg |

### 2. Bilayered

| Ingredients | Qty (mg/tablet) |
|---|---|
| Part - I | |
| Lamivudine | 60.00 |
| Nevirapine | 100.00 |
| Microcrystalline cellulose | 94.95 |
| Starch | 8.00 |
| Sodium starch glycollate | 10.00 |
| colourant | 0.05 |
| Talc | 3.00 |
| Magnesium stearate | 4.00 |
| Purified water | q.s |
| Part - II | |
| Stavudine | 12.00 |
| Mannitol | 81.00 |
| Hydroxypropyl cellulose | 4.00 |
| Colloidal silicon dioxide | 0.60 |
| Talc | 1.20 |
| Magnesium stearate | 1.20 |
| Total | 380.00 |

### b) Tablet in Tablet

| Ingredients | Qty (mg/tablet) |
|---|---|
| Tablet I | |
| Stavudine | 12.00 |
| Mannitol | 63.95 |
| Hydroxypropyl cellulose | 5.00 |
| Aerosil | 0.5 |
| Talc | 1.0 |
| Magnesium stearate | 2.5 |
| Colourant | 0.05 |
| | 85.00 |
| Tablet II | |
| Lamivudine | 60.00 |
| Nevirapine | 100.00 |
| Microcrystalline cellulose | 167.00 |
| Starch | 6.00 |
| Sodium starch glycollate | 8.0 |
| Purified water | q.s. |
| Talc | 1.0 |
| Magnesium stearate | 3.0 |
| | 345.00 |

1. A pharmaceutical composition comprising a single dosage unit containing lamivudine, stavudine, and nevirapine, or pharmaceutically acceptable salts or esters thereof, comprising 10-120 mg lamivudine, 1-30 mg stavudine and 25-170 mg nevirapine, wherein said stavudine is separated from said nevirapine and lamivudine within the composition.

2. The pharmaceutical composition according to claim 1, comprising 10-100 mg lamivudine, 1-25 mg stavudine and 25-170 mg nevirapine.

3. The pharmaceutical composition according to claim 1, comprising 60 mg lamivudine, 12 mg stavudine and 100 mg nevirapine.

4. The pharmaceutical composition according to claim 1, comprising 30 mg lamivudine, 6 mg stavudine and 50 mg nevirapine

5. The pharmaceutical composition according to claim 1, wherein the wt % of nevirapine in the composition is from times 0.75 to 2.0 times the wt % of lamivudine in the composition.

6. The pharmaceutical composition according to claim 1, wherein the wt % of lamivudine in the composition is from times 2 to 6 times the wt % of stavudine in the composition.

7. The pharmaceutical composition according to claim 6, wherein said dosage form is an oral dosage form.

8. The pharmaceutical composition according to claim 7, wherein said dosage form is a solid dosage form.

9. The pharmaceutical composition according to claim 7, wherein a barrier is provided between said stavudine and said nevirapine and lamivudine.

10. The pharmaceutical composition according to claim 7, wherein said stavudine is provided in the form of particles coated with a material to prevent contact between said stavudine and said nevirapine and lamivudine.

11. The pharmaceutical composition according to claim 7, comprising two layers, wherein the first layer contains said nevirapine and lamivudine in combination with a pharmaceutically acceptable carrier, and the second layer contains said stavudine in combination with a pharmaceutically acceptable carrier.

12. The pharmaceutical composition according to claim 7, comprising three layers, wherein the first layer contains said nevirapine and lamivudine in combination with a pharmaceutically acceptable carrier, the second layer contains said stavudine in combination with a pharmaceutically acceptable carrier, and the third layer is an inert layer containing at least one pharmaceutically acceptable excipient, wherein the third layer is disposed between the first and second layers.

13. The pharmaceutical composition according to claim 7, comprising a core and an outer layer surrounding the core, wherein the core contains said nevirapine and lamivudine in combination with a pharmaceutically acceptable carrier, and outer layer contains said stavudine in combination with a pharmaceutically acceptable carrier.

14. The pharmaceutical composition according to claim 7, comprising a core and an outer layer surrounding the core, wherein the core contains said stavudine in combination with a pharmaceutically acceptable carrier, and outer layer contains said nevirapine and lamivudine in combination with a pharmaceutically acceptable carrier.

15. The pharmaceutical composition according to claim 7, wherein said dosage form is a dispersible dosage form.

16. A method comprising using a lamivudine, nevirapine and stavudine in the manufacture of a medicament for treatment of a viral infection in a human, wherein said medicament is in the form of a single dosage unit contains 10-120 mg lamivudine, 1-30 mg stavudine and 25-170 mg nevirapine, and wherein said stavudine is separated from said nevirapine and lamivudine within the medicament.

17. The method according to claim 16, to treat a human under 16 years old.

18-24. (canceled)

25. The method according to claim 16, wherein the viral infection is a retroviral infection.

26. The method according to claim 16, wherein the viral infection is a HIV infection.

27. A method of treating a viral infection in a human, comprising administering to a human in need thereof an amount of a medicament in the form of a single dosage unit containing 10-120 mg lamivudine, 1-30 mg stavudine and 50-170 mg nevirapine at therapeutically acceptable intervals, wherein said stavudine is separated from said nevirapine and lamivudine within the medicament.

28. The method according to claim 27, wherein the viral infection is a retroviral infection.

29. The method according to claim 27, wherein the viral infection is a HIV infection.

30. The method according to claim 27, wherein the human is less than 16 years old.

31. A method of making a pharmaceutical composition comprising combining 10-120 mg lamivudine, 1-30 mg stavudine and 25-170 mg nevirapine into a single dosage form, with a pharmaceutically acceptable carrier, wherein said stavudine is separated from said nevirapine and lamivudine within the composition.

32. The method according to claim 31, comprising providing a pharmaceutically acceptable barrier between said stavudine and said nevirapine and lamivudine.

33. A method of making a bilayer pharmaceutical unit dosage form, comprising combining 10-120 mg lamivudine and 25-170 mg nevirapine with a pharmaceutically acceptable carrier to form a first layer, and combining 1-30 mg stavudine with a pharmaceutically acceptable carrier to form a second layer, and combining said first and second layers to form said dosage form.

34. A method of making a trilayer pharmaceutical unit dosage form, comprising combining 10-120 mg lamivudine and 25-170 mg nevirapine with a pharmaceutically acceptable carrier to form a first layer, combining 1-30 mg stavudine with a pharmaceutically acceptable carrier to form a second layer, forming a third layer comprising at least one pharmaceutically acceptable excipient, and combining said first, second and third layers to form said dosage form in which said third layer is disposed between the first and second layers.

35. A method of making a pharmaceutical unit dosage composition, comprising combining 10-120 mg lamivudine and 25-170 mg nevirapine with a pharmaceutically acceptable carrier to form a core, combining 1-30 mg stavudine with a pharmaceutically acceptable carrier to form a first layer on said core.

36. A method of making a pharmaceutical unit dosage composition, comprising combining 1-30 mg stavudine with a pharmaceutically acceptable carrier to form a core, and combining 10-120 mg lamivudine and 25-170 mg nevirapine with a pharmaceutically acceptable carrier to form a first layer on said core.

37. The method according to claim 35, wherein a pharmaceutically acceptable barrier layer is provided between the core and said first layer.

38. A pharmaceutical composition in the form of an oral unit dosage form comprising stavudine, lamivudine and nevirapine, wherein the lamivudine and nevirapine are provided in one layer of the oral dosage form, and the stavidine is provided in a separate layer.

39. A pharmaceutical composition in the form of an oral unit dosage form comprising stavudine, lamivudine and nevirapine, wherein said stavudine is provided in the form of particles coated with a material to prevent contact between said stavudine and said nevirapine and lamivudine.

40. The method according to claim 27, wherein the human is a child suffering from a viral infection and having a weight in the range 3 to under 6 kg, wherein the medicament is in the form of a single dosage unit containing 2 to 4 mg stavudine, 10 to 20 mg lamivudine and 20 to 30 mg nevirapine, preferably 3 mg stavudine, 15 mg lamivudine and 25 mg nevirapine, and wherein said stavudine is separated from said nevirapine and lamivudine within the medicament.

41. The method according to claim 27, wherein the human is a child suffering from a viral infection and having a weight in the range 6 to under 8 kg, wherein the medicament is in the form of a single dosage unit containing 5 to 7 mg stavudine, 20 to 40 mg lamivudine and 40 to 60 mg nevirapine, preferably 6 mg stavudine, 30 mg lamivudine and 50 mg nevirapine, and wherein said stavudine is separated from said nevirapine and lamivudine within the medicament.

42. The method according to claim 27, wherein the human is a child suffering from a viral infection and having a weight in the range 8 to under 10 kg, wherein the medicament is in the form of a single dosage unit containing 8 to 10 mg stavudine, 35 to 55 mg lamivudine and 65 to 85 mg nevirapine, preferably 9 mg stavudine, 45 mg lamivudine and 75 mg nevirapine, and wherein said stavudine is separated from said nevirapine and lamivudine within the medicament.

43. The method according to claim 27, wherein the human is a child suffering from a viral infection and having a weight in the range 10 to under 15 kg, wherein the medicament is in the form of a single dosage unit containing 11 to 13 mg stavudine, 50 to 70 mg lamivudine and 90 to 110 mg nevirapine, preferably 12 mg stavudine, 60 mg lamivudine and 100 mg nevirapine, and wherein said stavudine is separated from said nevirapine and lamivudine within the medicament.

44. The method according to claim 27, wherein the human is a child suffering from a viral infection and having a weight in the range 15 to under 20 kg, wherein the medicament is in the form of a single dosage unit containing 14 to 16 mg stavudine, 65 to 85 mg lamivudine and 115 to 135 mg nevirapine, preferably 15 mg stavudine, 75 mg lamivudine and 125 mg nevirapine, and wherein said stavudine is separated from said nevirapine and lamivudine within the medicament.

45. The method according to claim 27, wherein the human is a child suffering from a viral infection and having a weight in the range 20 to 24 kg, wherein the medicament is in the form of a single dosage unit containing 17 to 19 mg stavudine, 80 to 100 mg lamivudine and 140 to 160 mg nevirapine, preferably 18 mg stavudine, 90 mg lamivudine and 150 mg nevirapine, and wherein said stavudine is separated from said nevirapine and lamivudine within the medicament.

46. The method according to claim 27, wherein the human is a child suffering from a viral infection and having a weight in the range 25-29 kg, wherein the medicament is in the form of a single dosage unit containing 23 to 25 mg stavudine, 110 to 130 mg lamivudine and 190 to 210 mg nevirapine, preferably 24 mg stavudine, 120 mg lamivudine and 200 mg nevirapine, and wherein said stavudine is separated from said nevirapine and lamivudine within the medicament.
